(19) United States
(12) Patent Application Publication
(10) Pub. No.: US 2026/0069751 A1
(43) Pub. Date: Mar. 12, 2026

(54) NON-ENZYMATIC DEXTROSE SENSORS FOR DIALYSIS

(71) Applicant: Mozarc Medical US LLC, Minneapolis, MN (US)

(72) Inventors: Elisa Musella, Trento (IT); Piero Bruschi, Medolla (IT); Giuliano Giganti, Oppido Lucano (IT)

(21) Appl. No.: 19/322,233

(22) Filed: Sep. 8, 2025

Related U.S. Application Data
(60) Provisional application No. 63/693,391, filed on Sep. 11, 2024.

(51) Int. Cl. A61M 1/16; A61M 1/28; G01N 27/30; G01N 33/15

(52) U.S. Cl. CPC A61M 1/1607; A61M 1/1668; A61M 1/28; G01N 27/30; G01N 33/15; A61M 2205/3317

(57) ABSTRACT

In some examples, a sensor for detecting dextrose includes at least one working electrode including Cu and being configured to electro-oxidize dextrose in an aqueous solution. The sensor further includes at least one counter electrode. In some examples, a system includes a fluid line, a dextrose container, a peritoneal dialysis container, and the sensor. The fluid line is configured to transport water. The dextrose container is configured to introduce dextrose in the fluid line to generate a peritoneal dialysis composition comprising the water and dextrose. The peritoneal dialysis container is downstream of the dextrose container and configured to receive the peritoneal dialysis composition. The sensor may be configured to detect dextrose in the peritoneal dialysis composition.

### CROSS-REFERENCE TO RELATED APPLICATIONS

The present application claims the benefit of U.S. Provisional Application Ser. No. 63/693,391, filed Sep. 11, 2024, which is incorporated herein by reference in entirety.

### TECHNICAL FIELD

The present disclosure generally relates to dextrose sensors, for example, for dialysis.

### BACKGROUND

A dialysis fluid container may be used to store a fluid for dialysis, for example, water or an aqueous fluid. For example, the fluid may be used for peritoneal dialysis.

### SUMMARY

In general, the present disclosure describes sensors, systems, and techniques for detecting dextrose in dialysis fluid.

Dialysis fluid may be circulated within a body cavity of a patient, for example, to clean the peritoneal cavity, or for hemodialysis. Dialysis fluid may include dextrose and other components, for example, ionic components. Each component may have an associated concentration range and an associated tolerance. Sensors according to the present disclosure may be configured to detect dextrose in dialysis fluid, for example, without using enzymatic detection of dextrose. For example, an example sensor configured to detect dextrose may not include an enzyme responsive to dextrose. In some examples, a sensor includes at least one working electrode including copper (Cu), or a compound, alloy, or material including Cu.

Sensors according to the present disclosure may be cost-effective, and compact. Further, sensors according to the present disclosure may be configured to detect dextrose with little to no interference from other components of the dialysis fluid (e.g., from one or more ions present in the dialysis fluid).

In some examples, an example sensor for detecting dextrose includes at least one working electrode including Cu and being configured to electro-oxidize dextrose in an aqueous solution. The sensor further includes at least one counter electrode.

In some examples, an example system includes a fluid line, a dextrose container, and a peritoneal dialysis container. The fluid line is configured to transport water. The dextrose container is configured to introduce dextrose in the fluid line to generate a peritoneal dialysis composition (e.g., a fluid) comprising the water and dextrose. The peritoneal dialysis container is downstream of the dextrose container and configured to receive the peritoneal dialysis composition. The sensor includes at least one working electrode including Cu and being configured to electro-oxidize dextrose in the peritoneal dialysis solution. The sensor further includes at least one counter electrode. The sensor is configured to detect dextrose in the peritoneal dialysis composition.

The details of one or more examples of the techniques of this disclosure are set forth in the accompanying drawings and the description below. Other features, objects, and advantages of the techniques will be apparent from the description and drawings, and from the claims.

### BRIEF DESCRIPTION OF DRAWINGS

FIG. 1 is a block diagram illustrating an example system including an example sensor configured to detect dextrose in an aqueous solution.

FIG. 2 is a diagram illustrating a cross-sectional view of an example working electrode configured to detect dextrose.

FIG. 3 is a diagram illustrating a top view of an example sensor configured to detect dextrose in a solution and having an arcuate geometry.

FIG. 4 is a diagram illustrating a perspective view of an example sensor configured to detect dextrose in a solution and having a tubular geometry.

FIG. 5 is a diagram illustrating a top view of an example solid-state sensor configured to detect dextrose in a solution.

FIG. 6 is a diagram illustrating a top view of an example sensor chip configured to detect dextrose in a solution.

FIG. 7 is a schematic block diagram illustrating an example system including a dialysis container and a dialysis unit.

FIG. 8 is a chart illustrating a response time of an example sensor to dextrose detection with increase in a concentration of dextrose in a sample solution.

FIG. 9 is a chart illustrating a response time of an example sensor to dextrose detection in presence of ions and lactate in a sample solution.

FIG. 10 is a chart illustrating a linear response of an example sensor to dextrose concentration in a sample solution.

### DETAILED DESCRIPTION

The present disclosure generally relates to sensors, systems, and techniques for detecting dextrose, for example, in dialysis fluid.

Peritoneal dialysis is a home therapy treatment that requires relatively high storage volumes for dialysate bags. To save space and reduce logistical impact, the treatment may be performed using fluid prepared at the point of use, for example, using concentrates and water from a water for injection (WFI) system. Dialysis fluid for peritoneal dialysis applications is prepared with a predetermined concentration of dextrose (glucose), for example 0.55% w/w, 1.5% w/w, 2.5% w/w, or 4.25% w/w (28 mM, 76 mM, 126 mM, or 214 mM). Dialysis fluid for hemodialysis applications may typically include dextrose in a range from 0 to 25 mM. A certain deviation from a nominal concentration may be acceptable. For example, a tolerance or variation for dextrose concentration may be ±5% with respect to a nominal value.

One or more sensors may be used to determine a composition of a dialysis fluid. In some examples, a sensor is configured to detect dextrose in a dialysis fluid solution. For example, the sensor may be an electrochemical sensor. Electrochemical sensors are a class of chemical sensors in which an electrode is used as a transducer element in presence of an analyte. For example, in a stationary condition, a constant voltage is applied to a working electrode and current is detected between the working electrode and a counter electrode. The magnitude of the current is indicative of a concentration of the analyte. In other examples, a current is generated, and a resulting voltage indicative of the concentration of the analyte is detected.

In some examples, an example sensor for detecting dextrose includes at least one working electrode including Cu and being configured to electro-oxidize dextrose in an aqueous solution. The sensor further includes at least one counter electrode. In some examples, the sensor does not include an alkaline component. For example, the sensor may not include NaOH. In some examples, the sensor does not include a particulate component including copper. For example, the sensor may be absent of a particulate composition including metallic copper or an alloy including copper (e.g., a slurry including copper may be absent from the sensor). In some examples, the electrode potential associated with oxidation of dextrose is different from a potential associated with interfering components, for example, ionic components of dialysis fluid. Thus, ions present in dialysis fluid may not interfere with electro-oxidation of dextrose by the sensor.

While certain sensors are configured to detect a concentration of dextrose in blood or from a sample derived from blood (e.g., for monitoring diabetes), such sensors may not exhibit a linear current response in a range of dextrose concentration associated with dialysis fluid. For example, dextrose concentration in blood may be substantially lower than that in dialysis fluid, and sensors configured to detect lower concentrations of dextrose may not exhibit a linear response to higher concentrations of dextrose associated with dialysis fluid. Sensors configured to detect relatively low dextrose concentrations may be complex, for example, in composition and/or construction. The present sensors configured to detect relatively higher dextrose concentrations associated with dialysis fluid (e.g., greater than 0.01% w/w, or greater than 0.1% w/w, or greater than 0.5% w/w) may be simpler in composition and/or composition than other sensors, and thus may be less expensive and easier to manufacture, operate, and maintain. For example, no particular phase microstructure or nanostructure, or morphology may be required (e.g., for a Cu component), and a bulk, homogenous, or amorphous form of Cu or Cu salt or alloy may be used. Further, sensors configured to detect a concentration of dextrose in blood may be susceptible to interference from the chemistry of or the concentration of components associated with dialysis fluid, which may not be present in blood. Thus, sensors according to the present disclosure may exhibit greater accuracy and/or precision for detecting dextrose in dialysis fluid compared to sensors configured to detect dextrose in blood or other biological fluids.

Sensors, systems, and techniques according to the present disclosure may provide accurate and reliable measurement of dextrose concentration, for example, in aqueous solutions, or in dialysis fluid in particular. Sensors according to the present disclosure may be more compact, have a lower cost, and exhibit lower interference from other components of dialysis fluid than other sensors, for example, compared to other types of sensors, such as enzymatic sensors. Further, enzymatic sensors may exhibit a relatively short life (e.g., because of inactivation of enzyme over time, or because of oxygen-dependence of enzymes). In comparison, sensors according to the present disclosure may exhibit a relatively longer life, and continue to detect dextrose for an extended period.

For example, sensors, systems, and techniques according to the present disclosure may provide real-time or near-real time monitoring of dialysis fluid prepared at a point of use by introducing dextrose and other components in water. Real-time or near-real time monitoring may allow parametric release or mixing of components in fluid, which may reduce a post-production analytic burden after batch production of dialysis fluid. Such monitoring may also reduce or eliminate post-production quality control product hold time or product release time. Further, sensors, systems, and techniques according to the present disclosure may be implemented at different production volumes, including home or clinic dialysis fluid production.

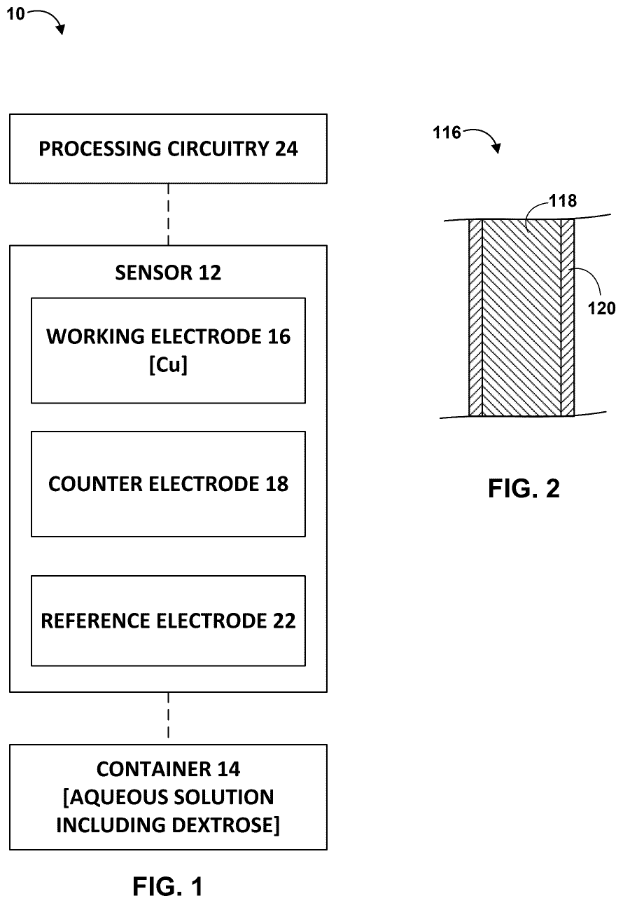

FIG. 1 is a block diagram illustrating an example system 10 including an example sensor 12 configured to detect dextrose in an aqueous solution. For example, system 10 may include a container 14 configured to receive a volume of the aqueous solution, and sensor 12 may be configured to at least partially contact the aqueous solution in container 14. The aqueous solution may be any solution including dextrose, for example, a dialysis fluid. In some examples, the aqueous solution is a peritoneal dialysis fluid. In addition to dextrose, the aqueous solution may include one or more ions, for example, sodium, potassium, magnesium, calcium, bicarbonate, or chloride.

Sensor 12 includes at least one working electrode 16. Working electrode 16 includes Cu (for example, one or more of metallic Cu, an alloy including Cu, or a compound or a salt including Cu). The Cu may be present in an amorphous phase, or in a homogenous phase, without requiring any particular phase or crystallinity of material. In some examples, an exterior surface of working electrode 16 includes a layer of oxidized Cu (e.g., CuO). The oxidized layer may be passively generated by oxidation of Cu with ambient oxygen, or actively generated by an appropriate process or treatment with an agent. The layer of oxidized Cu may act as a protective layer for working electrode 16. Working electrode 16 is configured to electro-oxidize dextrose in the aqueous solution. While sensor 12 is shown as having a single working electrode 16 in system 10 as illustrated in FIG. 1, in other examples, sensor 12 may include a plurality of working electrodes. In examples, in which sensor 12 includes a plurality of working electrodes 16, each working electrode of the plurality of working electrodes may be identical or different in one or more of shape, size, geometry, orientation, or composition.

Working electrode 16 may define any suitable surface area, for example, a working surface area at which dextrose is electro-oxidized. In some examples, working electrode 16 defines a surface area in a range from 1 mm² to 4 cm².

In some examples, working electrode 16 includes at least one of metallic Cu, an alloy including Cu, or a compound or a salt including Cu. For example, a bulk of working electrode 16, a portion of working electrode 16, or a coating applied to a substrate of working electrode 16 may include one or more of metallic Cu, the alloy including Cu, or the compound or the salt including Cu. In some examples, working electrode 16 consists of, or consists essentially of (e.g., except for minor impurities) metallic Cu. In some examples, working electrode 16 consists of, or consists essentially of (e.g., except for minor impurities), CuO. In some examples, working electrode 16 consists of, or consists essentially of (e.g., except for minor impurities), Cu(OH)₂. In some examples, working electrode 16 consists of, or consists essentially of, a mixture of CuO, Cu(OH)₂ and Cu.

FIG. 2 is a diagram illustrating a cross-sectional view of an example working electrode 116 configured to detect dextrose. Working electrode 116 may include a substrate 118 coated with a coating 120. Substrate 118 may have any suitable shape or geometry. For example, substrate 118 may be cylindrical, disk-shaped, cuboidal, spherical, or have any other suitable shape or cross-section. Coating 120 may have any suitable thickness. For example, coating 120 may have a thickness of at least 0.05 microns, at least 0.10 microns, at least 0.2 microns, at least 0.5 microns, at least 1 micron, at least 2 microns, or at least 5 microns. In some examples, coating 120 has a thickness of less than or equal to 10 microns, less than or equal to 5 microns, less than or equal to 2 microns, less than or equal to 1 micron, less than or equal to 0.5 microns, less than or equal to 0.2 microns, or less than or equal to 0.10 microns. In some examples, coating 120 has a thickness in a range from 0.05 microns to 10 microns, or from 0.10 microns to 10 microns, or from 1 micron to 10 microns, or from 5 micron to 10 microns, or from 0.05 microns to 5 microns, or from 0.05 microns to 2 microns, or from 1 micron to 5 microns.

Substrate 118 may include any suitable conductive metal or alloy. In some examples, substrate 118 includes Cu. In some such examples, substrate 118 includes metallic Cu, and coating 120 includes at least one of the alloy including Cu, or the compound or the salt including Cu. In other examples, substrate 118 does not include Cu in any form, and only coating 120 includes Cu. Coating 120 may include at least one of metallic Cu, the alloy including Cu, or the compound or the salt including Cu. For example, coating 120 may include CuO. In some examples, coating 120 consists of, or consists essentially of (e.g., except for minor impurities), CuO. In some examples, coating 120 may include Cu(OH)_{2.} For example, coating 120 may include, consist of, or consists essentially of a mixture of CuO, Cu(OH)₂, and Cu.

In some examples, coating 120 of working electrode 116 is configured to electro-oxidize dextrose.

Turning back to FIG. 1, sensor 12 further includes at least one counter electrode 18 (also known as an auxiliary electrode). Counter electrode 18 may include at least one of a metal or an alloy. In some examples, the metal or the alloy in the counter electrode includes at least one of Pt, graphite, Cu, Ag, or inox steel. A potential difference applied between working electrode 16 and counter electrode 18 generates a current that passes through the aqueous solution. Sensor 12 may further include electrical contacts, for example, in electrical communication with working electrode 16 and counter electrode 18, and the current may be sensed between the electrical contacts. The magnitude of the current may depend on and vary with concentration of dextrose in the aqueous solution, for example, via electro-oxidation of dextrose at a surface of working electrode 16. Thus, system 10 may be configured to detect the current, and generate a signal indicative of a magnitude of the current. In some examples, an area of counter electrode 18 A_{CE} is in a range with reference to an area of working electrode 16 A_{WE}, for example, as 0.5 A_{WE}≤A_{CE}≤3A_{WE}.

While sensor 12 is shown as having a single counter electrode 18 in system 10 as illustrated in FIG. 1, in other examples, sensor 12 may include a plurality of counter electrodes. In examples, in which sensor 12 includes a plurality of counter electrodes 18, each working electrode of the plurality of counter electrodes may be identical or different in one or more of shape, size, geometry, orientation, or composition. In some examples, each counter electrode of plurality of counter electrodes 18 is associated with a respective working electrode of plurality of working electrodes 16.

Sensor 12 may further include a housing configured to hold working electrode 16 and counter electrode 18. For example, the housing may maintain a predetermined spacing or orientation between working electrode 16 and counter electrode 18. The housing may include a glass, a polymer, a ceramic, a metal, or an alloy. The housing may provide suitable supports for at least one electrode (e.g., working electrode 16 or counter electrode 18), or for circuitry, and provide relatively smooth fluid dynamic conditions to promote accurate dextrose detection.

Sensor 12 may further include at least one reference electrode 22. Reference electrode 22 is configured to provide a reference potential such that a potential difference may be determined between working electrode 16 and reference electrode 22. Reference electrode 22 may include any suitable salt or salt system configured to generate a relatively stable reference potential. In some examples, reference electrode 22 includes at least one of Ag, AgCl, Hg, Hg₂Cl₂, Cu, or CuSO₄. For example, reference electrode 22 may include an Ag, Ag/AgCl, Hg/Hg₂Cl₂, or Cu/CuSO4 electrode system.

Sensor 12 may be configured to exhibit a linear response, or a substantially linear response, to a dextrose concentration in a predetermined range. For example, sensor 12 may be configured to exhibit a linear current response to a dextrose concentration greater than 0.01%, or greater than 0.1% w/w, or greater than 1% w/w. In some examples, sensor 12 is configured to exhibit a linear current response to a dextrose concentration of less than 20% w/w, or less than 10% w/w, or less than 7% w/w. In some examples, sensor 12 is configured to exhibit a linear current response to a dextrose concentration in a range from 0.01 % w/w to 20 % w/w, or from 0.01 % w/w to 10 % w/w, or from 0.1 % w/w to 10 % w/w, or from 0.1 % w/w to 7 % w/w. In some examples, sensor 12 is configured to exhibit a linear current response to a dextrose concentration in a range from 0.01% w/w to 7% w/w (from 0.5 mM to 380 mM).

Sensor 12 may further include processing circuitry 24. Processing circuitry 24 may be configured to detect an electrical parameter (for example, a current or a voltage potential between working electrode 16 and counter electrode 18) and generate a signal indicative of the electrical parameter. For example, a computing device (not shown in the figures) may be configured to receive the signal indicative of the electrical parameter, and determine, based on the electrical parameter, a dextrose concentration. In some examples, processing circuitry 24 is further configured to, based on the electrical parameter, generate the signal indicative of dextrose concentration.

Processing circuitry 24, as well as other processors, processing circuitry, controllers, control circuitry, and the like, described herein, may include any combination of integrated circuitry, discrete logic circuity, analog circuitry, such as one or more microprocessors, digital signal processors (DSPs), application specific integrated circuits (ASICs), or field-programmable gate arrays (FPGAs). In some examples, processing circuitry 24 includes multiple components, such as any combination of one or more microprocessors, one or more DSPs, one or more ASICs, or one or more FPGAs, as well as other discrete or integrated logic circuitry, and/or analog circuitry.

Processing circuitry 24 may be communicatively coupled to a memory that may store program instructions, such as software, which may include one or more program modules, which are executable by processing circuitry 24. When executed by processing circuitry 24, such program instructions may cause processing circuitry 24 and sensor 12 to provide the functionality ascribed to processing circuitry 24 and sensor 12 herein. The program instructions may be embodied in software and/or firmware. The memory, as well as other memories described herein, may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Processing circuitry 24 and the memory may be in a common housing, or be physically separate from each other.

Processing circuitry 24 may use any suitable technique to determine dextrose concentration based on the electric parameter detected by sensor 12. For example, processing circuitry 24 may implement a technique based on chronoamperometry, chronocoulometry, or chronopotentiometry. In case of chronoamperometric measurements, processing circuitry 24 may be configured apply a time-dependent potential (e.g., a square-wave potential) to working electrode 16. The current of working electrode 16, measured as a function of time, fluctuates according to the diffusion of an analyte (e.g., dextrose) from the aqueous solution toward a surface of working electrode 16. Processing circuitry 24 may thus be configured to implement chronoamperometry to measure current-time dependence for a diffusion-controlled process occurring at working electrode 16, which varies with analyte concentration. Chronoamperometry is a relatively sensitive technique, which does not require labeling of the analyte.

Processing circuitry 24 may implement chronocoulometry, which is similar to chronoamperometry except that processing circuitry 24 is further configured to integrate the current detected by sensor 12, and determine a variation of charge with time. The advantages of integration are that the signal increases with time, facilitating measurements towards the end of the transient, when the current is almost zero. Integration is effective in reducing signal noise and it is relatively easy to separate a capacitive charge from a faradaic charge. Further, processing circuitry 24 may implement constant-current chronopotentiometry, in which the processing circuitry 24 causes a constant current to be applied to working electrode 16, which causes the electroactive species to be reduced at a constant rate. The potential of working electrode 16 moves to values characteristic of a redox couple and varies with time as the concentration ratio changes at a surface of working electrode 16.

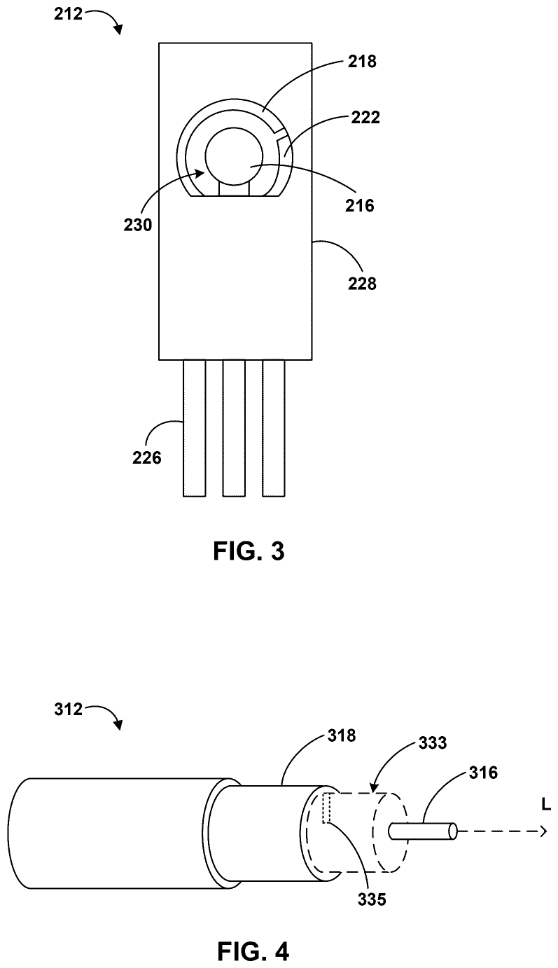

Thus, sensor 12 is configured to generate a signal indicative of dextrose concentration. Sensors according to the present disclosure may use any suitable geometric configuration for electrodes, for example, for a working electrode or a counter electrode. In some examples, as described with reference to FIGS. 3 and 4, at least one counter electrode at least partially circumferentially surrounds at least one working electrode.

FIG. 3 is a diagram illustrating a top view of an example sensor 212 configured to detect dextrose in a solution and having an arcuate geometry. For example, sensor 212 includes at least one counter electrode 218 extending along an arcuate path at least partially about at least one working electrode 216. The arcuate path may be circular, elliptical, or otherwise curved along any suitable path. Thus, counter electrode 218 may at least partially circumferentially surround working electrode 216, for example, along the arcuate path. The composition of working electrode 216 may be similar to that described with reference to working electrode 16 described with reference to FIG. 1. The composition of counter electrode 218 may be similar to that described with reference to counter electrode 18 described with reference to FIG. 1.

In some examples, working electrode 216 defines a disk, and counter electrode 218 extends along the arcuate path at least partially about the disk. Working electrode 216 may have any other suitable contour or shape, for example, a polygonal or a curved contour or shape. Counter electrode 218 may be spaced from working electrode 216 by any suitable spacing, for example, at least 0.1 cm, at least 0.2 cm, at least 0.5 cm, at least 0.7 cm, or at least 1 cm. Working electrode may have any suitable size, for example, having a maximum diameter in a range from 2 mm to 10 mm, from 2 mm to 5 mm, from 5 mm to 10 mm, from 7 mm to 10 mm, or from 2 mm to 7 mm. Counter electrode 218 may have any suitable width, for example, in a range from 2 mm to 30 mm, from 2 mm to 10 mm, from 2 mm to 20 mm, from 5 mm to 30 mm, from 5 mm to 20 mm, from 5 mm to 10 mm, from 10 mm to 30 mm, from 10 mm to 20 mm, or from 20 mm to 30 mm.

In some examples, sensor 212 further includes a reference electrode 222. The composition of reference electrode 222 may be similar to that described with reference to reference electrode 22 described with reference to FIG. 1. In some examples, reference electrode 222 at least partially extends about the same or similar arcuate path as counter electrode 218. In some such examples, working electrode 216 extends along a first segment of an arcuate path, and reference electrode 222 extends along a second segment of the arcuate path. Reference electrode 222 may be spaced from counter electrode 218 by any suitable spacing along the arcuate path. In some examples, reference electrode 222 extends along a path that is radially inward of counter electrode 218 toward working electrode 216. In other examples, reference electrode 222 extends along a path that is radially outward of counter electrode 218 away from working electrode 216.

Sensor 212 may further include a plurality of contacts 226, for example, being respectively electrically coupled with working electrode 216, counter electrode 218, and reference electrode 222. Sensor 212 may further include a housing 228 configured to hold one or more of working electrode 216, counter electrode 218, reference electrode 222, and contacts 226. In some such examples, housing 228 defines an opening 230 configured to allow dialysis fluid to contact working electrode 216 and counter electrode 218. Sensor 212 may be relatively compact, for example, compared to a sensor in which counter electrode 218 does not extend along an arcuate path.

FIG. 4 is a diagram illustrating a perspective view of an example sensor 312 configured to detect dextrose in a solution and having a tubular or coaxial geometry. For example, sensor 312 includes at least one working electrode 316 extending along a longitudinal axis L and at least one counter electrode 318 at least partially surrounding working electrode 316 along longitudinal axis L. In some such examples, counter electrode 318 is radially outward of working electrode 316, for example, as nesting cylindrical electrodes. For example, working electrode 316 may include a wire, and counter electrode 318 may define a tube surrounding the wire. The tube may be spaced from the wire by at least one spacer.

The composition of working electrode 316 may be similar to that described with reference to working electrode 16 described with reference to FIG. 1. The composition of counter electrode 318 may be similar to that described with reference to counter electrode 18 described with reference to FIG. 1. Working electrode 316 and counter electrode 318 may be separated by any suitable spacing transverse to or radial to longitudinal axis L, for example, in a range from 1 mm to 5 mm, from 1 mm to 3 mm, from 1 mm to 2 mm, from 2 mm to 5 mm, or from 3 mm to 5 mm. Working electrode 316 or counter electrode 318 may have any suitable thickness, for example, independently in a range from 0.50 mm to 2 mm, from 1 mm to 2 mm, or from 0.50 mm to 1 mm. Working electrode 316 or counter electrode 318 may have any suitable length, for example, independently in a range from 5 mm to 30 mm, from 10 mm to 30 mm, from 20 mm to 30 mm, from 5 mm to 10 mm, from 5 mm to 20 mm, or from 10 mm to 20 mm.

A region 333 between working electrode 316 and counter electrode 318 may be occupied by a volume of dialysis fluid (or any other analyte). In some examples, a radially outermost surface of counter electrode 318 is also exposed to or in contact with analyte. In some examples, working electrode 316 is spaced from counter electrode 318 by at least one spacer 335, for example, in region 333. Spacer 335 may include any suitable electrically insulating composition, or a composition that does not interfere with detection of dextrose by sensor 312. For example, spacer 335 may include a polymer, a ceramic, or a glass. Spacer 335 may have any suitable shape, size or geometry. For example, spacer 335 may be polyhedral or curved, for example, cuboidal, cylindrical, spherical, or ovoid. In some examples, working electrode 316 is spaced from counter electrode 318 by a plurality of spacers including spacer 335. In some examples, spacer 335 is a disk or a cylinder surrounding working electrode 316. In some such examples, each spacer of the plurality of spacers is identical. In other such examples, at least one spacer of the plurality of spacers differs from at least one another spacer of the plurality of spacers in one or more of size, composition, or shape.

In some examples, sensor 312 further includes a reference electrode (not shown in FIG. 4), which may be similar to reference electrode 22 described with reference to FIG. 1. However, in some examples, sensor 312 may not include a reference electrode. For example, electric field lines associated with working electrode 316 may be perpendicular to working electrode 316 (transverse to longitudinal axis L), thus reducing fringe fields and increasing a stability of a signal generated in response to dextrose. Thus, a reference electrode may not be necessary because the signal may be sufficiently stable due to reduction of fringe fields.

Sensor 312 may further include a plurality of contacts (not shown in FIG. 4), for example, being respectively electrically coupled with working electrode 316, counter electrode 318, and reference electrodes (if present). Sensor 312 may be relatively compact, for example, compared to non-tubular sensors.

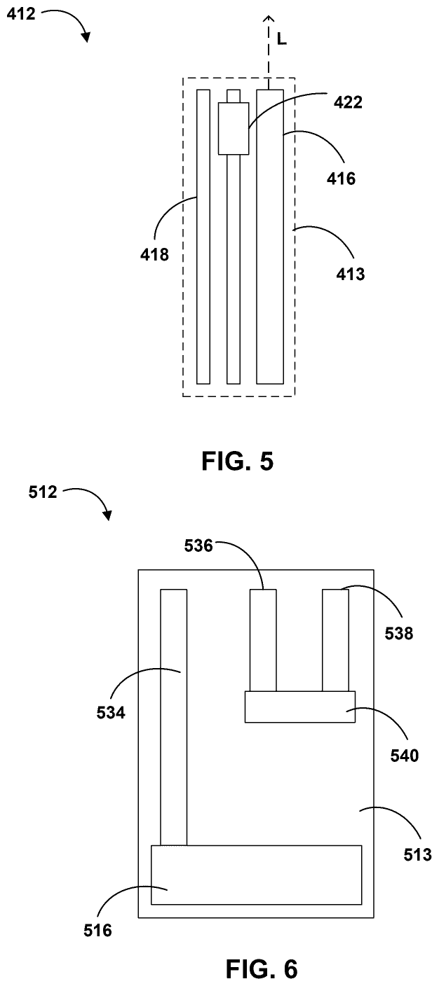

FIG. 5 is a diagram illustrating a top view of an example solid-state sensor 412 configured to detect dextrose in a solution. Sensor 412 may include a substrate 413 (e.g., an insulating substrate) on which electrodes are positioned. Substrate 413 may include a polymer, a ceramic, a glass, or a dielectric material. In some examples, sensor 412 includes at least one working electrode 416 extending along a longitudinal axis L, and at least one counter electrode 418 extending parallel to working electrode 416. For example, counter electrode 418 may be laterally spaced from working electrode 416, with counter electrode 418 and working electrode 416 extending along a same direction relative to longitudinal axis L. In some such examples, one or both of working electrode 416 or counter electrode 418 defines at least one of a disk, a spade, or a cylinder. For example, an end portion of electrode 416 or counter electrode 418 may define at least one of the disk, the spade, or the cylinder. The composition of working electrode 416 may be similar to that described with reference to working electrode 16 described with reference to FIG. 1. The composition of counter electrode 418 may be similar to that described with reference to counter electrode 18 described with reference to FIG. 1.

In some examples, sensor 412 further includes a reference electrode 422. In some examples, reference electrode 422 extends in a direction along longitudinal axis L and spaced from working electrode 416, for example, between working electrode 416 and counter electrode 418. A composition of reference electrode 422 may be similar to that of reference electrode 22 described with reference to FIG. 1. Sensor 412 may further include a plurality of contacts (not shown in FIG. 5), for example, being respectively electrically coupled with working electrode 416, counter electrode 418, and reference electrode 422 (if present). Sensor 412 may be relatively compact, for example, compared to non-solid state sensors.

FIG. 6 is a diagram illustrating a top view of an example sensor chip 512 configured to detect dextrose in a solution. Sensor chip 512 may be similar to sensor 412, but configured to function as a transistor. For example, sensor chip 512 may include a substrate 513 (e.g., a dielectric substrate), and electrodes of sensor chip 512 may respectively be coupled to electrical contacts defining a gate 534, a source 536, and a drain 538. For example, gate 534 may be electrically coupled to a working electrode 516, and source 536 may interact with drain 538 via a channel 540. Working electrode 516 may include a Cu-based substrate or Cu-based film, and have a composition similar to that described with reference to working electrode 516 of FIG. 1. One or more of gate 534, source 536, or drain 538 may include metal or alloy contacts. The metal or alloy contacts may be coated with a coating, for example, polydimethylsiloxane (PDMS). The metal or alloy contacts may include any suitable metal component, for example Cu, Ag, or Au. Gate 534 may be configured to drive current between source 536 and drain 538. Thus, a gate signal delivered to gate 534 may be used to control a current between source 536 and drain 538. The current is proportional to the gate signal and to dextrose concentration.

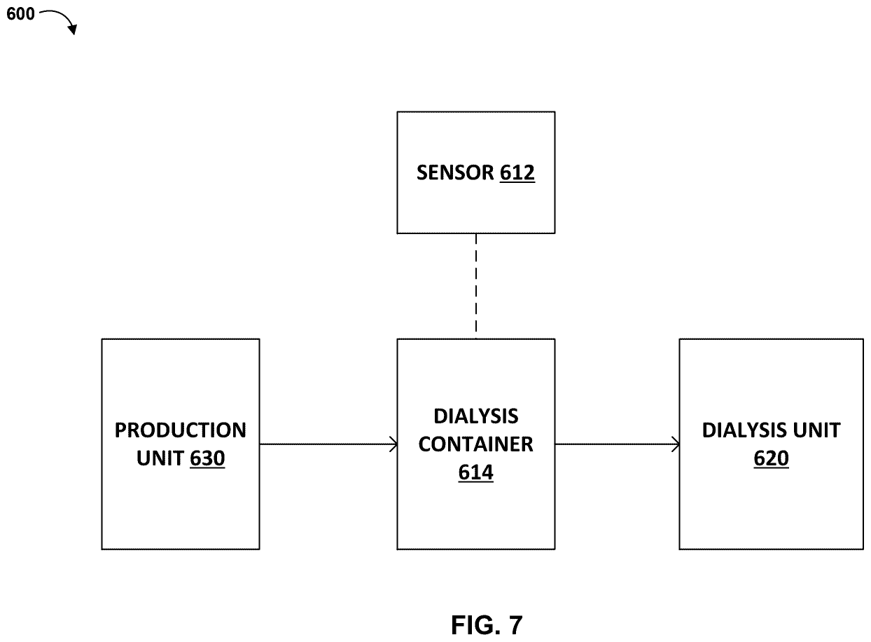

FIG. 7 is a schematic block diagram illustrating an example system 600 including a dialysis container 614 and a dialysis unit 620. Dialysis unit 620 is configured to be fluidically coupled to a peritoneal cavity of a patient, for example, to perform peritoneal dialysis. However, system 600 may be used to perform any dialysis procedure. For example, a fluid for dialysis may be transferred from dialysis container 614 to dialysis unit 620, which may in turn supply the fluid to irrigate the peritoneal cavity of the patient. System 600 may further include a production unit 630 configured to supply or replenish fluid in dialysis container 614. For example, production unit 630 may include a preparator, a dextrose source, and at least one ion source. Further, production unit 630 may include or be fluidically coupled to a water source. Production unit 630 may be configured to introduce dextrose from the dextrose source and at least one from the at least one ion source into water from the water source to prepare a dialysis fluid. Dialysis container 614 may receive the dialysis fluid from production unit 630.

System 600 further includes a sensor 612 configured to detect dextrose in dialysis fluid, for example, in dialysis container 614, or in any fluid line or component of system 600.

Sensor 612 may include any sensor according to the present disclosure. Sensor 612 may be configured to generate a signal indicative of dextrose concentration in dialysis fluid in system 600. In some examples, system 600 further includes processing circuitry, for example, processing circuitry 24 described with reference to system 10, configured to receive the signal indicative of dextrose concentration from sensor 512.

Processing circuitry 24 may be configured to generate an output indicative of the dextrose concentration, and/or control one or more components of system 600 (e.g., one or more of production unit 630, dialysis container 614, dialysis unit 620, or at least one valve coupled to at least one component of system 600) in response to the dextrose concentration. For example, processing circuitry 24 may be configured to cause dialysis unit 620 to allow dialysis fluid to flow toward a patient in response to determining that the dextrose concentration is in a nominal range, and to prevent flow of dialysis fluid toward the patient in response to determining that the dextrose concentration exceeds the nominal range. Further, processing circuitry 24 may be configured to cause production unit 630 to introduce additional dextrose into dialysis fluid in response to determining that the dextrose concentration is lower than the nominal range, and to introduce water into dialysis fluid in response to determining that the dextrose concentration is higher than the nominal range.

Sensors according to the present disclosure may be fabricated using any suitable technique. For example, one or more electrodes may be fabricated by at least one of screen printing, ink-jet printing, chemical synthesis (for example co-precipitation or hydrothermal synthesis), or electro-synthesis technique. Electrodes may be treated with an annealing process, for example, annealing at a temperature up to 400° C.

In some examples, a technique for forming a component including Cu (e.g., a working electrode or a counter electrode) includes electrodeposition of Cu from an aqueous solution including Cu. For example, the aqueous solution may include any suitable salt including Cu (for example, a chloride salt or a nitrate salt of Cu). Electrodeposition with an appropriate deposition voltage may be used to deposit Cu (e.g., as metallic Cu, or a salt or alloy of Cu) in a predetermined pattern. Cu may be applied using a copper-based ink, instead of or in addition to electrodeposition. For example, the copper-based ink may include a salt or particles (e.g., particles include CuO) including Cu dissolved or suspended in a solvent or carrier, and the ink may be applied in a predetermined pattern. The solvent or carrier may be removed, for example, by evaporation or dehydration at ambient or elevated temperatures, to leave the Cu in the predetermined pattern. In some examples, the component including Cu may be formed using chemical vapor deposition or physical vapor deposition.

### EXAMPLE

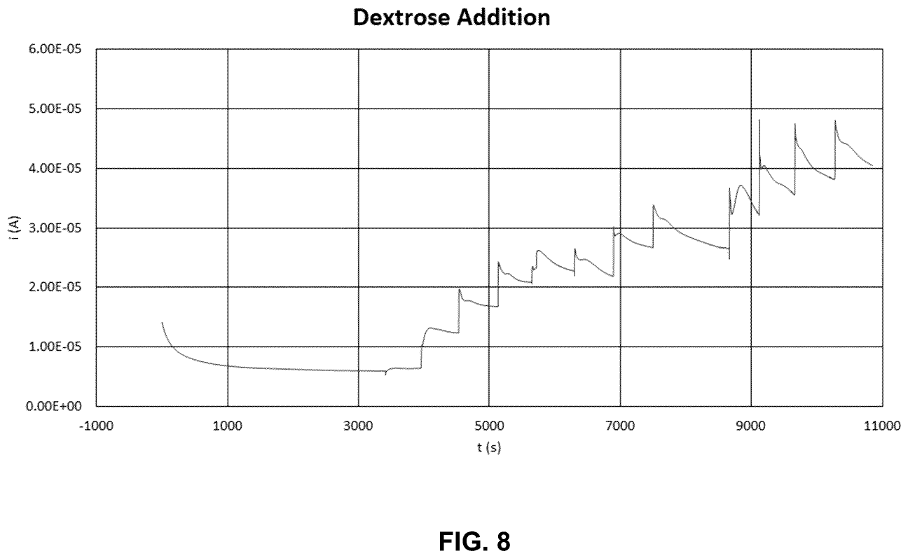

An example sensor was prepared, including a working electrode including Cu, and a reference electrode. The sensor included a layer of copper electrodeposited from aqueous solution of copper salt onto an electrode support. The copper electrodeposition and the electrochemical characterizations of the support were carried out in a three-electrode cell, and all potentials were controlled by a potentiostat (Interface 1010E from Gamry Instruments, Westminster, PA). A glassy carbon electrode (4 mm of diameter) was used as the working electrode, while a Ag/AgCl and a Pt gauze were used as the reference and counter electrodes, respectively. The electrodeposition was performed by applying a constant potential of −0.4 V with respect to Ag/AgCl for 500 s. The electrolytic solution included 0.15 M Cu(NO₃)₂·3H₂O and 0.70 M KNO₃. The CuₓO_{y}—Cu catalyst was achieved by applying a constant potential of −0.4 V vs Ag/AgCl for 600 s in 0.5 M KOH aqueous solution, obtaining an oxide layer on top of the copper. The as-prepared sensor was used to detect dextrose in a sample solution (peritoneal dialysis fluid). The response of the sensor to dextrose under different conditions is described with reference to FIGS. 8 to 10.

FIG. 8 is a chart illustrating a response time of an example sensor to dextrose detection with increase in a concentration of dextrose in a sample solution.

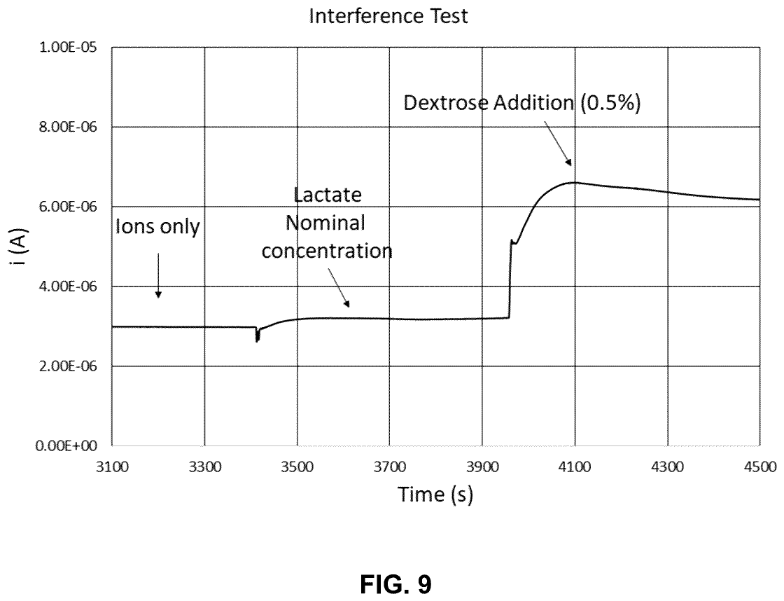

FIG. 9 is a chart illustrating a response time of an example sensor to dextrose detection in presence of ions and lactate in a sample solution.

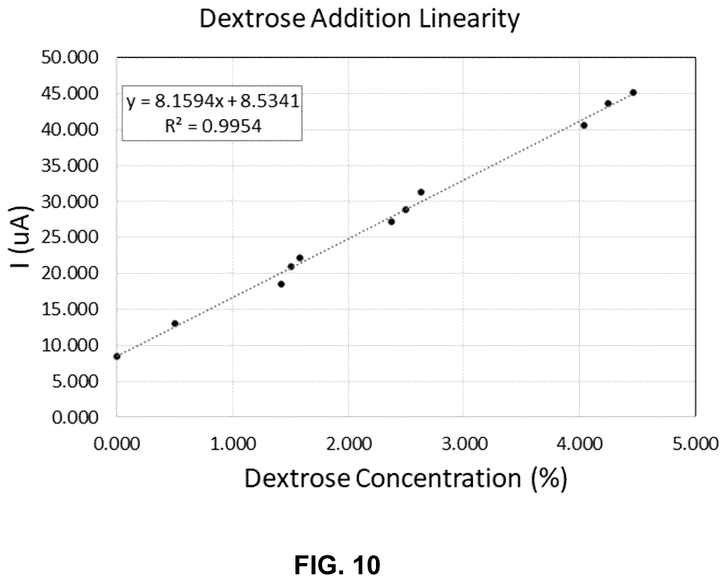

FIG. 10 is a chart illustrating a linear response of an example sensor to dextrose concentration in a sample solution.

While articles, systems, and techniques according to the present disclosure may be used for peritoneal dialysis, they may also be used for other dialysis applications, for example, hemodialysis, or for any application in which dextrose is sensed.

Various examples have been described. These and other examples are within the scope of the following claims.

What is claimed is:

1. A sensor for detecting dextrose, the sensor comprising:
at least one working electrode comprising Cu and configured to electro-oxidize dextrose in an aqueous solution; and
at least one counter electrode.

2. The sensor of claim 1, wherein the at least one working electrode comprises at least one of metallic Cu, CuO, or Cu(OH)₂.

3. The sensor of claim 1, wherein the at least one working electrode comprises a substrate coated with a coating comprising CuO.

4. The sensor of claim 3, wherein the substrate comprises metallic Cu.

5. The sensor of claim 3, wherein the coating consists essentially of CuO.

6. The sensor of claim 1, wherein the sensor is configured to exhibit a linear current response to a dextrose concentration in a range from 0.01% to 7% w/w.

7. The sensor of claim 1, wherein the at least one working electrode defines a surface area in a range from 1 mm² to 4 cm².

8. The sensor of claim 1, wherein the aqueous solution is a dialysis fluid.

9. The sensor of claim 1, wherein the at least one counter electrode comprises at least one of Pt, graphite, Cu, Ag, or inox steel.

10. The sensor of claim 1, further comprising at least one reference electrode.

11. The sensor of claim 10, wherein the at least one reference electrode comprises at least one of Ag, AgCl, Hg, Hg₂Cl₂, Cu, or CuSO₄.

12. The sensor of claim 1, wherein the at least one counter electrode at least partially circumferentially surrounds the at least one working electrode.

13. The sensor of claim 12, wherein the at least one working electrode defines a disk, and wherein the at least one counter electrode extends along an arcuate path about the at least one working electrode.

14. The sensor of claim 1, wherein the at least one working electrode extends along a longitudinal axis, and wherein the counter electrode surrounds the at least one working electrode along the longitudinal axis.

15. The sensor of claim 14, wherein the at least one working electrode comprises a wire, and wherein the counter electrode defines a tube surrounding the wire.

16. The sensor of claim 14, wherein the at least one working electrode is spaced from the at least one counter electrode by at least one spacer.

17. The sensor of claim 1, wherein the at least one working electrode extends along a longitudinal axis, and wherein the at least one counter electrode extends parallel to the at least one working electrode.

18. The sensor of claim 17, the at least one working electrode defines at least one of a disk, a spade, or a cylinder.

19. The sensor of claim 1, wherein the sensor is absent NaOH.

20. A system comprising:
a fluid line configured to transport water;
a dextrose container configured to introduce dextrose in the fluid line to generate a peritoneal dialysis composition comprising water and dextrose;
a peritoneal dialysis container downstream of the dextrose container and configured to receive the peritoneal dialysis composition; and
a sensor fluidically coupled to the fluid line and configured to determine a concentration of dextrose in the peritoneal dialysis composition,
wherein the sensor comprises:
at least one working electrode comprising Cu and configured to electro-oxidize dextrose in the peritoneal dialysis composition; and
at least one counter electrode.
